(19) United States
(12) Patent Application Publication
(10) Pub. No.: US 2003/0007960 A1
(43) Pub. Date: Jan. 9, 2003

(54) Oral methods of treatment

(76) Inventors: John Lezdey, Indian Rocks Beach, FL (US); K. Anne Kronis, Tampa, FL (US); Darren Lezdey, Indian Rocks Beach, FL (US)

Correspondence Address: John Lezdey & Associates, 4625 EAST BAY DRIVE, SUITE302, Clearwater, FL, 33764, US

(21) Appl. No.: 09/957,012

(22) Filed: Sep. 20, 2001

Related U.S. Application Data
(63) Continuation-in-part of application No. 09/885,654, filed on Jun. 20, 2001.

(51) Int. Cl. A61K 38/43; A61K 31/573

(52) U.S. Cl. 424/94.1; 514/179

(57) ABSTRACT

A method is provided for the treatment of non-pulmonary diseases and injuries by the oral administration of a stabilized protease inhibitor that transgresses the gastrointestinal tract. Crystalline, crosslinked and polyethylene glycol adducts can be used as well as coated protease inhibitors and coated stabilized protease inhibitors.

### RELATED APPLICATIONS

This application is a continuation-in-part of application Ser. No. 09/885,654

### FIELD OF THE INVENTION

The present invention relates to the treatment of non-bronchial diseases characterized by elevated matrix metallo-proteinases and the release of inflammatory medicators from mast cells by the oral administration of proteins that possess an inhibitory activity against matrix metallo-proteinases, tumor necrosis factor alpha (TNF-α) and/or elastase.

More particularly, it is provided an oral medicament that transgresses the gastrointestinal pathway to deliver an effective amount of medicament to the blood stream.

### BACKGROUND OF THE INVENTION

Matrix metallo-proteinases (MMPs) are secreted by connective tissue cells. The MMPs are elevated in collagen related diseases such as rheumatoid arthritis, scleraderma and intersticial cystitis. Cigarette smoke induces an enzyme that enhances MMP release in the lungs. Also, smoker's skin is degraded by MMPs.

MMPs include collagenase (MMP1), gellatinase (MMP2), and stromelysin (MMP3). Tumor invasiveness has been attributed to the activity of MMP1.

Mast cells when degraded during disease or injury release the mediators of inflammation which include elastase, cathepsin G and TNF-α.

In rheumatoid arthritis there is an elevation of TFN-α, elastase, cathepsin G, and MMP1. The combination of elastase and cathepsin G has been shown to cause tissue destruction to the same degree as collagenase.

The treatment of any of the diseases by oral administration of protease inhibitors for non-bronchial disease has not been successful because of the degradation of the proteins in the gastrointestinal tract. Also, it was previously believed by those in the art that protease inhibitors had no effect on collagen related diseases. Oral administration of alphal-antitrypsin in the treatment of cystic fibrosis was found to be ineffective.

The prior art treatment of collagen related disease such as rheumatoid arthritis did not relate to the inhibition of collagenase or TNF-α which are the principal destructive members in the disease.

There have been many methods proposed to stabilize proteins so that they can be administered orally. Crosslinking the protein, forming conjugates, and crystallization have all been suggested in some instances.

U.S. Pat. No. 5,880,255 to Delgade et al., which is herein incorporated by reference, discloses the preparation of polyethylene glycol (PEG)-protein which process can be used to prepare the adducts of the invention that transgress the gastrointestinal tract in the invention.

U.S. Pat. No. 6,004,549 to Reichert et al, which is herein incorporated by reference, disclose the preparation of crystalline protein and polyethylene glycol or vegetable oil, which can be used in the practice of the invention.

U.S. Pat. No. 5,554,730, which is herein incorporated by reference, discloses a process for preparing polysaccharide-protein conjugates.

Each of alpha 1-antitrypsin, secretory leucocyte protease inhibitor and alpha2-macroglobulin has been disclosed by Lezdey et al as having anti-viral characteristics and they have also been effective against certain bacteria, especially Pseudomonas.

U.S. Pat. No. 6,140,475 to Margolin, which is herein incorporated by reference, discloses the preparation or crosslinked crystalline proteins. The process disclosed can be used for the preparation of the compositions used in the present invention.

Protein crystals can be grown by the controlled crystallization of protein out of aqueous solution or aqueous solution-containing organic solvents. Conditions to be controlled include, for example, the rate of evaporation of solvent, the presence of appropriate co-solutes and buffers, pH and temperature. A comprehensive review of the various factors affecting the crystallization of proteins has been published by McPherson, Methods Enzymol., 114, pp. 112-20 (1985).

McPherson and Gilliland, J. Crystal Growth, 90, pp. 51-59 (1988) have compiled comprehensive lists of proteins and nucleic acids that have been crystallized, as well as the conditions under which they were crystallized. A compendium of crystals and crystallization recipes, as well as a repository of coordinates of solved protein and nucleic acid structures, is maintained by the Protein Data Bank at the Brookhaven National Laboratory [http://www.pdb.bnl.gov; Bernstein et al., J Mol. Biol., 112, pp. 535-42 (1997)]. These references can be used to determine the conditions necessary for crystallization of a protein, as a prelude to the formation of an appropriate crosslinked protein crystal, and can guide the crystallization strategy for other proteins. Alternatively, an intelligent trial and error search strategy can, in most instances, produce suitable crystallization conditions for many proteins, provided that an acceptable level of purity can be achieved for them [see, e.g., C. W. Cater, Jr. and C. W. Carter, J. Biol. Chem., 254 pp. 12219-23 (1979)].

### SUMMARY OF THE INVENTION

The invention provides for the oral delivery of a protein that can be used in the treatment of diseases characterized by increased MMPs, TNFα, and elastase. More particularly, there is provided a composition for oral administration which transgresses the gastrointestinal tract to deliver a drug to the blood.

Accordingly, there is provided the crosslinked, crystalline, conjugate or encapsulated proteins selected from the group consisting of alpha 1-antitrypsin, alpha 2-macroglobulin and secretory leucocyte protease inhibitor in a form so as to transgress the gastrointestinal tract and pass the protease inhibitors through the gut into the bloodstream.

The compositions of the invention can also include anti-oxidants such as glutathione, catalase, superoxide dismutase, mannitol, methionine, and the like, especially in connection with smokers to prevent degredation of the skin.

Among the diseases which may be treated with the protease inhibitors, there are included rheumatoid arthritis, scleraderma, intersticial cystitis, eczema, psoriasis, wound healing and the like.

The use of alpha 1-antitrypsin is especially useful in the treatment of the various inflammatory conditions involving TNF-α and elastase, which are released from mast cells. Alpha 1-antitrypsin irreversibly binds with MMP1.

The combination of other serine protease inhibitors with alpha 1-antitrypsin such as secretory leucocyte protease inhibitor and/or alpha 2-macroglobulin provides a broader spectrum for treating associated symptoms of inflammation or the immune related diseases.

The genetic mutagenesis of the serine protease inhibitors may be performed utilizing conventional techniques associated with other genes without affecting the utility of the compound. In AIDS, it is believed that the disease injures the liver and reduces the body's production of AAT so as to result in the skin lesions and pulmonary complications.

It is an object of the invention to provide an anti-inflammatory composition which can be used as a prophylaxis and/or treatment of existing inflammatory and MMP related diseases, particularly, dermatological and intestinal diseases.

It is another object of the invention to provide a composition for smokers which can prevent skin degradation arising from tobacco use.

It is a further object of the invention to provide an anti-inflammatory composition for non-pulmonary diseases, which is well tolerated by the human body and is free of side effects.

It is yet a further object of the invention to provide a composition for use in the treatment of gastrointestinal diseases, including ulcers and Crohn's disease.

It is also an object of the invention to elevate the serum level of a patient with the protease inhibitors to combat viral and bacterial infections.

### DETAILED DESCRIPTION OF THE PREFERRED EMBODIMENTS

The object of the present invention can be achieved by the oral administration of protease inhibitors in suitable pharmaceutical form to patients suffering from non-pulmonary inflammatory conditions resulting from an excess of MMPs, TNF-α and/or elastase. Additionally, the compositions can be used to treat viral and bacterial infections.

The present invention provides a pharmaceutical composition, which comprises the protease inhibitor alone or in combination with anti-oxidants.

Normally between 100 and 500 mg of the compositions of the invention will be administered each day of treatment (to an average 70 kg adult) to control the disease. Lower amounts may be administered to prevent the occurrence of the condition.

In the treatment of chronic cases of inflammatory conditions wherein the cells express the proteases, TNF-α and elastase such as in the case of rheumatoid arthritis, the patient is typically administered a daily dose of 200 mg of the protease inhibitor compound for the average adult. The treatment is continued for a period of time until there is a reversal of the biochemical abnormalities in serum and tissues that characterizes the disorder. For use in the prevention of the disease, the drug should be administered orally on a daily basis.

Patients with severe rheumatoid arthritis usually include a corticosteroid in their regimen. The corticosteroid can be taken together or separately with the protease inhibitor.

According to one embodiment of the present invention, there is provided an orally deliverable formulation enabling the therapeutic delivery of a protease inhibitor of interest to through the stomach to be released in the gut so as to pass into the bloods streams. One formulation method requires the production of a multiparticulate dosage core particle. The multiparticulate dosage core particle is made up of three components, the total weight of the three components in dry form defining a batch size.

One of the three components referred to above is polyethylene glycol (PEG). An aqueous PEG solution is prepared. In preferred embodiments, the dry weight of the PEG component represents from about 2.5% to about 15% of the batch size (weight/weight), and the water component of the aqueous PEG solution represents approximately 30-60% of the batch size (weight/weight). Preferably, PEG 4,000-PEG 8,000 is employed in connection with the present formulation. For intersticial cystitis the composition must mainly transgress the intestinal tract where the bile acids are formed.

Macrophages cause the release of a metalloproteinase that solubilizes many extracellular matrix proteins, including elastase. These metalloproteinases generate monocyte chemotatic activity. Thus, macrophage-mediated destruction after increased macrophage activity is directly related to the metalloproteinase, which can result in destruction of tissue. Inactivation of the metalloproteinasese at an early stage is advisable to prevent the large proteinase-anti-proteinase imbalance that leads to tissue destruction.

A homogenous mixture of the protease inhibitor and microcrystalline cellulose, both in dry form, can also be prepared. In preferred embodiments of this method, the protease inhibitor represents from about 50% to about 95% of the batch size (weight/weight). Microcrystalline cellulose comprises from about 2.5% to about 35% of the batch size (weight/weight). This composition can be used in treating interstitial cystitis, Crohn's disease and ulcers.

The adducts of the invention metabolize to the natural or recombinant protease inhibitors. Therefore, any form of oral administration of the protease inhibitors, which transgresses the gastrointestinal tract can be utilized. It is understood that there are many methods of stabilizing the protease inhibitors to pass through the gastrointestinal tract. The stabilization is also useful to provide a longer shelf life to the protease inhibitors.

It is further understood that the amount of protease inhibitor to be administered is dependant upon the age, weight and severity of condition. The compositions are especially useful in the early stages of the disease so as to maintain an early protease-antiprotease balance.

The formulations are suitable for treating viral diseases such as STD's, for example human papalloma virus (HPV) and herpes.

The following examples are the only representations of preferred methods of producing the stabilized protease inhibitors. The anti-oxidants that can be used in the invention include glutathione, catalase, mannitol and the like.

### EXAMPLE 1

A tablet containing conjugates having a clear coating is prepared as follows.

A. Tablets are prepared on a TF-MINI roller compactor apparatus sold by Vector Corp., which comprise 50 mg secretory leucocyte protease inhibitor and 50 mg of alpha 1-antitrypsin in each tablet. The tablets are formed with the following inactive ingredients: mannitol, cellulose hydroxypropyl methyl cellulose, polyethylene glycol, sodium starch glycolate and starch.

B. The tablets are coated in a Vector Hi-Coater perforated pan coating system with the following formulation

| Ingredients | WT | |
|---|---|---|
| Eudagrit ® | 100.0 | g |
| Glidant (talc) | 7.5 | g |
| Plasticizer | 3.0 | g |
| Water | 92.0 | g |

Eudagrit® comprises 30 g of polymer and the remainder water.

The glidant, plasticizer and water are mixed in a separate vessel using a high shear mixer. This is then added to the Eudagrit® dispersion and is mixed at low shear.

The glidant may vary from 25 to 100% by weight based on the solids of Eudagrit®. The plasticizer may vary from 10 to 12% by weight based on the amount of polymer solids.

Glidants which may be used include silica, glycerol monostearate and natural kaolin.

Plasticizers which may be used include citrate esters, PEG, tuarin and DBS. The plasticizers are usually utilized in the range of about 10 to 12% by weight based on the solid polymers. The mannitol also serves as the antioxidant.

The coated tablet can be used to transgress the gastrointestinal tract for use in the treatment of dermatological diseases.

The composition can be used to prevent or treat the onset of eczema.

In lieu of SLPI there may be used AAT or alpha 2-macroglobulin.

This type of composition is particularly suited for including a corticosteroid such as beta-methasone to prevent interaction between the drugs since the corticosteroid can be first coated and formed into pellets for use in combination in the treatment of diseases in which interleukin 4 is involved.

### EXAMPLE 2

### Crystalization of alpha 1-antitrypsin

Following the procedure of U.S. Pat. No. 6,140,475, 1 gram of alpha 1-antitrypsin (PROLASTIN) is dissolved in 100 ml saline solution. 1 gram of celite is dissolved in 100 ml of distilled deionized water and filtered. Solid calcium acetate is added to a concentration on 5 mM Ca(CH₃C00)₂. The pH is adjusted to pH 5.5 with concentrated acetic acid. 200 ml of 30% solution of PEG-8000 is added to the mixture and cooled overnight and the resulting crystals filtered.

The crystals can be crosslinked using glutanaldehyde or pursuant to the procedure in Augervante Chemie Inl Ed. Eugl 35 p. 2056 (1996).

The resulting crystals can be formulated into a tablet and coated so as to transgress the gastrointestinal tract in the treatment of non-pulmonary diseases.

Alternatively, a crosslinked product can be used with a coating.

What is claimed is

1. A method for treatment of mammals suffering from a non-pulmonary disease which comprises orally administering an effective amount of a composition of a protein, which has been stabilized so as to transgress the gastrointestinal tract, said protein being the conjugate, crosslinked or crystalline adduct of a member selected from the group consisting of alpha 1-antitrypsin, secretory leucocyte protease inhibitor and alpha 2-macroglobulin.

2. The method of claim 1 including an antioxidant.

3. The method of claim 2 wherein said antioxidant is selected from the group consisting of glutathione, catalase and mannitol.

4. The method of claim 1 wherein said protein is a polyethylene glycol-alpha 1-antitrypsin adduct.

5. The method of claim 1 wherein said protein in a polyethylene glycol-secretory leucocyte protease inhibitor adduct.

6. The method of claim 1 wherein said protease inhibitors are crystalline.

7. The method of claim 1 wherein said protease inhibitors are crosslinked.

8. The method of claim 1 wherein said protease inhibitors are conjugated.

9. The method of claim 8 wherein said protease inhibitor is alpha 1-antitrypsin conjugated with dextran or polyethylene glycol.

10. The method of claim 1 wherein said composition includes a corticosteroid.

11. The method of claim 1 including the step of separately administering orally a corticosteroid.

12. The method of claim 1 wherein said disease is eczema.

13. The method of claim 1 wherein said disease is intersticial cystitis.

14. The method of claim 1 wherein said disease is rheumatoid arthritis.

15. The method for treatment of mammals suffering from a disease characterized by matrix metallo-proteinases which comprises orally administering an effective amount of a composition of a protein, which has been stabilized so as to transgress the gastrointestinal tract, said protein being the conjugate, crosslinked or crystalline adduct of a member selected from the group consisting of alpha 1-antitrypsin, secretory leucocyte protease inhibitor and alpha 2-macroglobulin.

16. The method of claim 15 wherein said protein is a polyethylene glycol alpha 1-antitrypsin adduct.

17. The method of claim 15 wherein said protein is a polyethylene glycol-secretory leucocyte protease inhibitor adduct.

18. The method of claim 15 wherein said protease inhibitors are crystalline.

19. The method of claims 15 wherein said protease inhibitors are crosslinked.

20. The method of claim 15 wherein said protease inhibitors are conjugated.
